(19) United States
(12) Patent Application Publication
(10) Pub. No.: US 2012/0134948 A1
(43) Pub. Date: May 31, 2012

(54) ANTIMICROBIAL ETHER GUANIDINES

(75) Inventors: Oliver Springer, Wesel (DE); Peter Muss, Essen (DE); Peter Lersch, Dinslaken (DE); Ursula Maczkiewitz, Essen (DE); Mike Farwick, Essen (DE)

(73) Assignee: EVONIK GOLDSCHMIDT GMBH, Essen (DE)

(21) Appl. No.: 13/389,115

(22) PCT Filed: Jul. 19, 2010
(86) PCT No.: PCT/EP2010/060404

§ 371 Date: Feb. 6, 2012

(30) Foreign Application Priority Data
Aug. 31, 2009 (DE) 102009029010.9

(51) Int. Cl. A61K 8/43; A61K 31/155; A61Q 15/00; A01P 1/00; A61P 31/00; A61P 31/04; A01N 37/52; C07C 279/08; A61P 17/10

(52) U.S. Cl. 424/65; 564/240; 514/634

(57) ABSTRACT

The present invention relates to the use of at least one ether guanidine of the general formula (I)

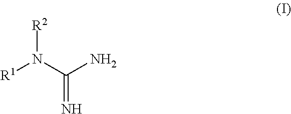

and/or of a salt or hydrate thereof, in which
R¹═—CH₂—CH₂—CH₂—O—R³, where R³ is a linear or branched hydrocarbon radical having 6 to 22 carbon atoms, and
R²═H or an optionally branched hydrocarbon radical which has 1 to 22 C atoms and which optionally comprises double bonds,
for reducing the growth of microorganisms,
and to cosmetic and pharmaceutical formulations comprising at least one ether guanidine of the general formulae (I).

### FIELD OF THE INVENTION

The invention relates to the use of ether guanidines as antimicrobial active substance in, in particular, cosmetic and/or pharmaceutical preparations.

Antimicrobial active substances are used widely in cosmetic deodorants, anti-dandruff and anti-acne formulations, footcare products, feminine hygiene products, oral hygiene products and dental care products, and in disinfectants.

Body odor arises mainly when sweat, which is initially odorless, is decomposed on the skin by microorganisms. It is only the microbial degradation products which cause the unpleasant odor of sweat. The latter arises in particular where there is a high density of sweat glands and in addition a high density of odor-generating microorganisms such as Corynebacterium xerosis, such as, for example, under the armpit.

Certain skin diseases, too, are related to the excessive growth of undesired microorganisms on the skin. Thus, for example, acne is caused by the uncontrolled proliferation of the anaerobic skin bacterium Propionibacterium acnes, inter alia. Soor or candidosis is triggered by Candida albicans. Dandruff is connected, inter alia, with the fungus Malassezia furfur.

In the field of oral hygiene, microorganisms play an important role for example in the development of caries and dental plaque. Among others, for example, Streptococcus mutans is responsible for the development of caries.

### PRIOR ART

The use of antimicrobial active substance in cosmetic formulations, in particular in cosmetic deodorants, anti-dandruff shampoos and anti-acne creams, is well known from the prior art.

The spectrum of, for example, deodorant products ranges from solid formulations via liquid or cream-like O/W- or W/O-emulsions to aqueous, alcoholic or oil-comprising liquid systems. Furthermore, such preparations cover a large pH range, which can reach from approximately 2 to approximately 12.

As the result, biocidal active substances must not only meet high requirements regarding stability (in particular, no hydrolysis or alcoholysis should take place under the abovementioned conditions), but also in respect of its activity, which should be as constant as possible over the entire pH range of the products which are possible.

Substances which are used are, for example, triclosan(5-chloro-2-(2,4-dichlorophenoxy)phenol), which have an antimicrobial activity against a broad spectrum of microorganisms. Owing to its broad-spectrum activity, triclosan has a disadvantageous effect on the microflora of the skin; moreover, its specific mode of action means that there is a risk of resistances developing.

It is known from the prior art that guanidines and ether guanidines have antimicrobial activity. Thus, for example, GB 1112307 describes ether guanidines as active substances against fungi and bacteria as early as in 1965. However, it provides no examples for a use in corresponding cosmetic and/or pharmaceutical preparations.

DE 19527313 describes guanidine derivatives as components of skin cosmetics. However, nothing is mentioned about an antimicrobial activity of the guanidine derivatives.

DE 1107215 describes biocidal guanidine compounds which are said to distinguish themselves by a wide range of activity and a good bactericidal activity. Furthermore, it is described that the compositions can be used on their own, in solution and in ointment bases and creams.

It was an object of the invention to provide active substances which have antimicrobial properties and which are suitable for being incorporated into cosmetic and pharmaceutical formulations.

### DESCRIPTION OF THE INVENTION

Surprisingly, it has been found that the ether guanidines described hereinbelow likewise have very good antimicrobial properties and can be employed in a very simple manner in cosmetic and/or pharmaceutical preparations. Furthermore, the compounds are distinguished by a selective control of undesired microorganisms, in particular those which colonize the skin.

The present invention therefore relates to the use of compounds of the general formula (I) as described in claim 1 for reducing the growth of microorganisms, in particular on a surface, and to formulations comprising these compounds.

The use according to the invention of the ether guanidines has the advantage that they exhibit not only good stability, but also good formulation properties. Moreover, they even cause a pronounced effect at low use concentrations, are not toxic, are tolerated very well by the skin and the hair, are very compatible with other constituents and can be formulated in a problem-free manner.

An advantage of the present invention is that the compounds are stable in apolar and polar (in particular alcoholic or aqueous) systems since no hydrolysis or alcoholysis take place under the conditions required.

Yet another advantage is that the activity is constant to a high degree over the entire pH range of the cosmetic formulations which are possible.

A further advantage is that the compounds are well tolerated by the skin.

All the percentages given (%) are percent by weight, unless otherwise specified.

Where the use takes the form of a use on a live species, the use according to the invention is exclusively a cosmetic and a nontherapeutic use.

When the term “ether guanidines” is used hereinbelow, it is understood as meaning not only the ether guanidines themselves, but also their salts or hydrates.

The expression “formulations” comprises what are known as “leave-on” products such as creams, lotions, pump sprays or aerosol sprays, wipes, deodorant sticks and roll-on formulations; “rinse-off” products such as shower gels, liquid soaps, oral hygiene products such as mouthwash solutions or toothpastes; cleaners such as washing-up liquid, domestic cleaners (floor cleaners, kitchen cleaners, bath cleaners), but without imposing any limitation.

Surprisingly, it has been found that ether guanidines which have a specific carbon chain length demonstrate the desired antimicrobial properties.

The present invention therefore relates to the use of at least one ether guanidine of the general formula (I)

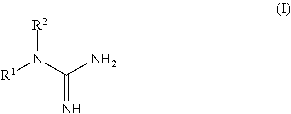

and/or of a salt or hydrate thereof, in which

R¹═—CH₂—CH₂—CH₂—O—R³, where R³ is a linear or branched hydrocarbon radical having 6 to 22 carbon atoms, and

R²═H or an optionally branched hydrocarbon radical which has 1 to 22 C atoms and which optionally comprises double bonds,

for reducing the growth of microorganisms, in particular on a surface.

In this context, the growth of microorganisms can be reduced either by simply inhibiting the growth and the accompanying natural death of the microorganisms or by destroying the organisms by the compound of the general formula (I), where actively destroying the microorganisms is preferred.

The at least one ether guanidine of the general formula (I) is preferably used in a pharmaceutical form as a cosmetic and pharmaceutical formulation, with anti-dandruff products being excepted.

A preferred embodiment is the use according to the invention of the at least one ether guanidine of the general formula (I) in deodorant formulations.

A further preferred embodiment is the use according to the invention of the at least one ether guanidine of the general formula (I) in oral hygiene formulations such as, for example, toothpastes and mouthwashes.

A further preferred embodiment is the use according to the invention of the at least one ether guanidine of the general formula (I) in feminine hygiene formulations.

A further preferred embodiment is the use according to the invention of the at least one ether guanidine of the general formula (I) in anti-acne formulations

A further preferred embodiment is the use according to the invention of the at least one ether guanidine of the general formula (I) in cleaners.

Ether guanidines of the general formula (I) which are preferably used in accordance with the invention are those in which the radicals R² are exclusively hydrogen atoms.

It is preferred to use the ether guanidines of the general formula (I) in which the hydrocarbon radical R³ having 12 to 18 carbon atoms, and in particular those in which the hydrocarbon radical R³ having 12 to 15 carbon atoms

It is obvious that it is possible to use mixtures of two or more compounds of the general formula (I) according to the invention.

It is preferred in accordance with the invention that mixtures of a plurality of ether guanidines of the general formula (I) are used, in particular mixtures of those mentioned hereinabove as being preferred.

It is preferred to use mixtures in which not only hydrocarbon radicals R³ which have 12 carbon atoms, but also those which have 13, 14 and 15 carbon atoms, and that these are present in each case in an amount of from 10 to 50% by weight, preferably 15 to 40% by weight and especially preferably in an amount of from 20 to 30% by weight, based on the weight of all hydrocarbon radicals R³.

Especially preferred are those ether guanidines of the general formula (I) which are obtained by guanidylation of the amine PA-19 from Tomah Products

The ether guanidines can be used in accordance with the invention as a salt, for example as the salt of an organic or inorganic acid. When used as a salt, the ether guanidines can be used in accordance with the invention for example as the salt of at least one of the acids selected from the group of the substituted or unsubstituted, preferably unsubstituted, carboxylic acids (mono-, di- and polycarboxylic acids), such as, for example, formic acid, acetic acid, propionic acid, butanoic acid, isobutanoic acid, hexanoic acid, heptanoic acid, octanoic acid, caprylic acid, nonanoic acid, decanoic acid, capric acid, undecanoic acid, lauric acid, myristic acid, palmitic acid, stearic acid, arachic acid, behenic acid, cyclopentanecarboxylic acid, cyclohexanecarboxylic acid, acrylic acid, methacrylic acid, vinylacetic acid, isocrotonic acid, crotonic acid, 2-/3-/4-pentanoic acid, 2-/3-/4-/5-hexenoic acid, lauroleic acid, myristoleic acid, palmitoleic acid, oleic acid, gadoleic acid, sorbic acid, linoleic acid, linolenic acid, pivalic acid, ethoxyacetic acid, phenylacetic acid, glycolic acid, lactic acid, cinnamic acid, sorbic acid, nicotinic acid, urocanic acid, pyrrolidonecarboxylic acid, 2-ethylhexanoic acid, oxalic acid, glycolic acid, malic acid, malonic acid, succinic acid, tartaric acid, glutaric acid, citric acid, adipic acid, pimelic acid, suberic acid, azelaic acid, sebacic acid, maleic acid, fumaric acid, benzoic acid, o-/m-/p-toluic acid, o-/m-/p-hydroxybenzoic acid, salicylic acid, 3-/4-hydroxybenzoic acid, phthalic acid, terephthalic acid, or their fully or partially hydrogenated derivatives such as hexahydro- or tetrahydrophthalic acid; amino acids such as, for example, glycine, alanine, beta-alanine, valine, leucine, phenylalanine, tyrosine, serine, threonine, methionine, cysteine, cystine, proline, hydroxyproline, pipecolic acid, tryptophan, aspartic acid, asparagine, glutamic acid, glutamine, lysine, histidine, ornithine, arginine, or aminobenzoic acid; alkylsulfonic acids such as, for example, methanesulfonic acid or trifluoromethanesulfonic acid; arylsulfonic acids such as, for example, benzensulfonic acid or p-toluenesulfonic acid; or inorganic acids such as, for example, carbonic acid, phosphoric acid, hydrochloric acid, hydrobromic acid, hydriodic acid, hydrofluoric acid, perchloric acid, nitric acid or sulfuric acid, and mixtures thereof preferably the salt of lactic acid, tartaric acid, acetic acid, sulfuric acid or hydrochloric acid, and preferably as the salt of hydrochloric acid.

The preparation of the ether guanidines can be carried out in a manner known per se. In particular, the preparation of the ether guanidines can be carried out in a similar manner as the preparation of alkylguanidines, by guanylating the corresponding amines. The preparation of alkylguanidines is described, for example in DE-C-506 282. In the process, alkyl amines are guanidylated with cyanamide in alcoholic solution in the presence of a protonic acid. The products are obtained as crystalline salts. The preparation of the ether guanidines used in accordance with the invention can be carried out analogously by reacting ether amines with cyanamide in alcoholic solution in the presence of a protonic acid.

The preparation of the ether guanidines used in accordance with the invention can also be carried out by reacting the ether amines with guanidylating agents other than cyanamide. A list of other guanidylating agents and methods is found, inter alia, in EP 1 462 463, Ullmann's Encyclopedia of Industrial Chemistry “Guanidine and Derivatives” chapter 2.4, or Houben-Weyl, E 4, 608-624.

The ether amines to be employed can be obtained in a simple manner by reacting corresponding alcohols R³—OH, with R³ having the abovementioned meaning, with acrylonitrile as shown in the reaction scheme hereinbelow:

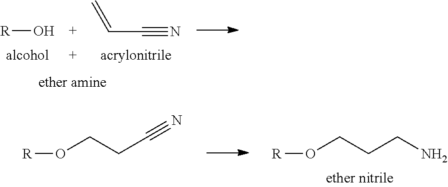

A detailed description of the preparation of such ether amines can be found for example in EP 1 219 597.

Ether amines are commercially available products and are sold, inter alia, by Tomah Products (USA) under the trade name Tomamine® and by Evonik Degussa GmbH under the trade name Adogen®.

By the use according to the invention of the ether guanidines, it is possible, for example, to reduce the number of microorganisms in a solution, in particular in a cosmetic or pharmaceutical formulation. This corresponds to the basic concept of a preservative. However, the use according to the invention is preferably carried on a surface.

Preferably, the surface in the use according to the invention is the surface of a human or animal body part, in particular of the skin, the hair or the teeth.

As a rule, a large number of undesired microorganisms is present on these surfaces, such as, for example, bacteria and fungi (including yeasts), specifically Corynebacterium xerosis, Propionibacterium acnes, Staphylococcus epidermidis, Candida albicans, Streptococcus mutans, Malassezia furfur.

Thus, a use wherein the microorganisms are Gram-positive, in particular coryneform, bacteria is preferred.

In the event that the surface is the surface of a live species, the compounds of the general formula (I) are preferably used in accordance with the invention in the form of a formulation. Further preferred uses are in particular those uses which make use of the formulations described hereinbelow, in particular in the form of the preferred formulations described hereinbelow.

The invention furthermore relates to a formulation, in particular to a cosmetic or pharmaceutical formulation, comprising at least one ether guanidine of the general formula (I)

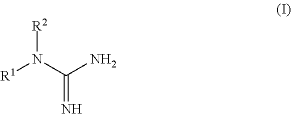

and/or of a salt and/or hydrate thereof, in which

R¹═—CH₂—CH₂—CH₂—O—R³, where R³ is a linear or branched hydrocarbon radical having 6 to 22 carbon atoms, and

R²═H or an optionally branched hydrocarbon radical which has 1 to 22 C atoms and which optionally comprises double bonds.

It is preferred that hair treatment products and hair aftercare products, anti-dandruff products, in particular shampoos, conditioners and serums, in particular for the hair, are excepted from these formulations according to the invention.

Formulations which are preferred in accordance with the invention comprise ether guanidines of the general formula (I) which are mentioned as being preferred hereinabove, or preferred mixtures of those.

Formulations according to the invention preferably comprise from 0.1 to 15% by weight of at least one compound of the general formula (I), based on the total formulation.

Suitable application forms of the cosmetic and/or pharmaceutical products are, for example, creams, lotions, solutions, liquids, emulsions such as W/O, O/W, PIT emulsions (emulsions referred to as PIT emulsions according to the phase-inversion theory), microemulsions and multiple emulsions, gels, sprays, aerosols and aerosol foams.

The cosmetic or pharmaceutical formulations according to the invention may comprise for example at least one additional component selected from the group of the
emollients,
emulsifiers,
thickeners/viscosity regulators/stabilizers,
UV sunscreens,
antioxidants,
hydrotropes (or polvols),
solids and fillers,
film formers,
pearlescent additives,
deodorants and antitranspirant active ingredients
insect repellents,
self-tan ingredients,
preservatives,
conditioners,
perfumes,
colorants,
cosmetic active ingredients,
care additives,
superfatting agents,
solvents.

Substances which can be employed as examples of the representatives of the individual groups are known to the skilled worker and can be found for example in the German application DE 102008001788.4. This patent application is therewith incorporated as reference and is therefore part of the disclosure.

As regards further facultative components, and as regards the amounts used of these components, the relevant manuals known to the person skilled in the art, for example K. Schrader, “Grundlagen und Rezepturen der Kosmetika [Basics and formulas of cosmetics]”, 2^{nd} edition, pages 329 to 341, Huthig Buch Verlag Heidelberg, are expressly referred to.

The amount of the respective additions depend on the intended use.

Typical formulas for the respective uses are known prior art and can be found for example in the leaflets of the manufacturers of the respective excipients and active substances. These existing formulas can, as a rule, be followed without modification. If required, however, the desired modifications for adaptation and optimization purposes may be carried out without problems by simple experiments.

The ether guanidines of the general formula (I) can be employed in a multiplicity of formulations for use in domestic settings, industry, pharmacy and cosmetics. They are especially suitable as effective components in deodorants, which may be present in the form of aerosol sprays, pump sprays, roll-on formulations, deodorant sticks, W/O or O/W emulsions (for example creams or lotions) or in wipes. Active substances/active substance combinations which can be used concomitantly in these formulations are those known from the prior art such as, for example, triclosan, ethylhexylglycerin, aluminum chlorohydrate, aluminum zirconium tetrachlorohydrex GLY, aluminum zirconium pentachlorohydrate, farnesol, poly-glycerol caprinate or caprylate, triethyl citrate, penta(carboxymethyl)diethylenetriamine (pentetic acid), pentylene glycol, propylene glycol, ethanol, zinc ricinoleate, cyclodextrins or zinc oxide.

However, the application is not limited to the use in deodorants, but can be advantageous wherever the control of microorganisms or of their growth is desired such as, for example, in feminine hygiene articles, anti-acne products which may be present in the form of the customary leave-on or rinse-off formulations, such as, for example, creams, lotions, wash solutions, wipes and similar formulations.

For anti-acne products, the substances according to the invention can, if appropriate, also be employed in combination with known anti-acne active substances such as, for example, dibenzoyl peroxide, salicylic acid, phytosphingosine, tretinoin, isotretinoin or plant extracts. Likewise, it is possible to use, in the case of anti-dandruff products, combinations with known anti-dandruff active substances such as, for example, climbazol, zinc pyrethion, selenium compounds (for example selenium sulfide), piroctone olamine (octopirox) or plant extracts.

The substances according to the invention can also be used in the field of oral hygiene products, lending themselves in particular to the use in oral rinse solutions or in toothpastes.

They can also be employed in combination with other active substances which have gaps in their activity in fields in which the ether guanidines are active. Thus, it is possible to reduce the use of preservatives or, indeed, to completely dispense with traditional preservatives.

The cosmetic and/or pharmaceutical formulations according to the invention which feature the ether guanidines of the general formula (I) can additionally comprise from 0 to 10% by weight, preferably from 0.1 to 7.5% by weight, of one or more emulsifiers, from 0 to 10% by weight, preferably from 0.1 to 7.5% by weight, of one or more consistency regulators, from 0 to 10% by weight, preferably from 0.1 to 7,5% by weight, of one or more, preferably cationic, surfactants and/or polymers comprising one or more quaternary ammonium group(s) and/or from 0 to 20% by weight, preferably from 0.1 to 17.5% by weight, of one or more cosmetic oils or emollients and, if appropriate, customary adjuvants and additives in usual concentrations. The remainder may be water, for example (water to 100% by weight).

The invention furthermore relates to a method of reducing the growth of microorganisms, comprising the steps
a) providing a cosmetic or pharmaceutical formulation according to the invention
b) applying the formulation to the surface to be treated, in particular skin, hair or teeth, in an effective amount
c) leaving the formulation on the treated surface for a sufficient period to ensure that the growth of the microorganisms is reduced, and
d) if appropriate washing off or rinsing off the formulation.

The examples mentioned hereinbelow describe the present invention by way of example; however, the invention, whose scope results from the entire description and the claims, shall not be limited to the use forms mentioned in the examples.

### EXAMPLES

### Example 1

### Synthesis of C12-15-oxypropylguanidinium chloride

271 g of Tomamine® PA-19 were dissolved, in 100 ml of n-butanol, with stirring. Thereafter, 86.4 g of hydro-chloric acid (38% strength) were added, and the mixture was warmed slowly. After the reaction temperature of 95° C. had been reached, a solution of 42 g of cyanamide in 240 ml of n-butanol was added dropwise over a period of 1 h, and stirring was continued at 95° C. for 2 hours. The solvent was subsequently stripped off under reduced pressure (approx. 1-2 mbar). The crude product was recrystallized from 325 ml of ethyl acetate and crystallized at 10° C. The end product was present as a colorless crystalline powder.

C₁₂₋₁₅-Oxypropylguanidinium chloride: ¹³C NMR, 100 MHz, CD₃OD, 25° C.: δ=158.6 (1C, C_{guanidinium gr.}) 72.0 (1C, OCH₂) , 68.4 (1C, OCH₂), 39.7 (1C, NHCH₂), 33.0 (1C, CH₂), 30.8 (5-8C, CH₂), 30.6 (1C, CH₂), 30.4 (1C, CH₂), 30.1 (1C, CH₂), 27.2 (1C, CH₂), 23.7 (1C, CH₂), 14.7 (1C, CH₃)

### Example 2

### Antimicrobial Activity

The test microorganisms were inoculated from a strain culture into an agar-containing Caso medium (Merck, Darmstadt, Germany) and incubated for 24 h to 48 hours at 25 to 37° C., depending on the microorganism. A Caso slanted agar tube was inoculated with this preculture and again incubated under identical conditions.

To prepare the microorganism suspension to be tested, the microorganisms were washed off with 5 ml of phosphate-buffer saline (PBS, 50 mM potassium phosphate pH6, 100 mM NaCl), made up to 12.5 ml with PBS and resuspended by vortexing. To carry out the following test, this microorganism suspension was diluted to a microbial count of from 2U+5 to 8U+5.

The products to be tested made as a stock solution; they contained 10 g of ethanol, 0.68 g of macrogol glycerol hydroxystearate 40 (Cremophor® RH40, Omikron, Neckarwestheim, Germany), 1 g of the active substance to be tested and 38.32 g of double-distilled water. This solution was mixed with the microorganism suspension at a ratio of 1:1 in the test and gave an active substance concentration in the test of 1%. If it was intended to test a lower active substance concentration than 1%, the stock solution of the active substance was made up at a correspondingly lower concentration. The substances tested were the ether guanidine of example 1, and double distilled water by way of negative control.

To carry out the test, 1 ml-Eppendorf cubes were filled with 500 μl of microorganism suspension and with 500 μl stock solution and the contents were mixed carefully. After 20 min, 1 h and 3 h, respectively, 10 μl were removed from the tubes, and the live microbial count (colony-forming units, CFU) were determined by plating out various dilution of these test mixtures on Caso agar.

A pronounced antimicrobial activity of ether guanidine was observed. The microbial count in the negative controls remained unchanged.

| Conc. of | | CFU/ml | | | |
|---|---|---|---|---|---|
| Ex. 1 | Microorganism | 0 min | 20 min | 60 min | 180 min |
| 1% | Candida albicans | 3.9E+5 | <2.0E+2 | <2.0E+2 | <2.0E+2 |
| 0.1% | Candida albicans | 3.9E+5 | 5.5E+5 | 4.3E+4 | <2.0E+2 |
| 0.01% | Candida albicans | 3.9E+5 | 4.7E+5 | 2.2E+5 | 6.1E+4 |
| 1% | Streptococcus mutans | 3.2E+5 | <2.0E+2 | <2.0E+2 | <2.0E+2 |
| 1% | Corynebacterium xerosis | 1.7E+5 | <2.0E+2 | <2.0E+2 | <2.0E+2 |
| 1% | Propionibacterium acnes | 2.0E+5 | <2.0E+2 | <2.0E+2 | <2.0E+2 |
| 1% | Malassezia furfur | 1.4E+5 | <2.0E+2 | <2.0E+2 | <2.0E+2 |
| 1% | Staphylococcus epidermidis | 1.2E+5 | <2.0E+2 | <2.0E+2 | <2.0E+2 |

### Example 3

### Anti-Acne Face Cleansing Lotion

| INCI name | % by weight |
|---|---|
| PEG-40 hydrogenated castor oil | 1.5% |
| fragrance | 0.1% |
| PEG-6 caprylic/capric glycerides | 1.5% |
| ethanol | 5.0% |
| capryl/capramidopropylbetaine | 5.2% |
| allantoin | 0.3% |
| water | 86.2% |
| ether guanidine of example 1 | 0.2% |
| preservative | q.s. |

Preparation: PEG-40 hydrogenated castor oil was initially introduced and warmed to approx. 40° C. until it was clear. Thereafter, the remaining components of the formula were added step by step with stirring, without warming.

### Example 4

### Moisturizing Anti-Acne Face Cleansing Gel

| INCI name | % by weight |
|---|---|
| water | 55.2% |
| glycerol | 10.0% |
| carbomer | 1.6% |
| ether guanidine of example 1 | 0.3% |
| sodium laureth sulfate, 28% | 21.4% |
| TEGO betaine F50 | 5.3% |
| fragrance | 0.2% |
| sodium hydroxide, 10% | 6.0% |
| preservative | q.s. |

Preparation: Water and glycerol were mixed. Then, the carbomer was added slowly with vigorous stirring, and stirring was continued until swelling was complete. Thereafter, the remaining components of the formula were added step by step.

### Example 5

### Anti-Acne Face Cream

| INCI name | % by weight |
|---|---|
| phase A | |
| polyglyceryl-3-methylglucose distearate | 3.0% |
| glyceryl stearate | 2.0% |
| stearyl alcohol | 1.0% |
| cyclopentasiloxane | 6.0% |
| ethylhexyl stearate | 5.0% |
| diethylhexyl carbonate | 3.0% |
| phytosphingosine | 0.1% |
| phase B | |
| glycerol | 3.0% |
| ether guanidine of example 1 | 0.2% |
| water | 76.5% |
| phase C | |
| fragrance | 0.2% |
| preservative | q.s. |

Preparation: Phase A and B were warmed separately to 80° C. Thereafter, phase B was added to A and the mixture was homogenized. Then, the emulsion was cooled to approximately 30° C., with stirring. The fragrance was added during the cooling process at below 40° C.,

### Example 6

### Anti-Acne Face Lotion

| INCI name | % by weight |
|---|---|
| phase A | |
| glyceryl stearate citrate | 1.5% |
| cetearyl alcohol | 1.0% |
| caprylic/capric triglyceride | 8.5% |
| myristyl myristate | 4.0% |
| tocopheryl acetate | 1.0% |
| almond (Prunus dulcis) oil | 1.0% |
| phase B | |
| glycerol | 5.0% |
| water | 76.9% |
| ether guanidine of example 1 | 0.1% |
| phase C | |
| carbomer | 0.2% |
| ethylhexyl stearate | 0.8% |
| phase D | |
| sodium hydroxide, 10% | q.s. |
| phase Z | |
| preservative | q.s. |
| fragrance | q.s. |

Preparation: Phase A and B were warmed separately to 80° C. Thereafter, phase B was added to A and the mixture was homogenized. Then, the emulsion was cooled to 60° C., with stirring, and phase C was added. The mixture was rehomogenized briefly. The emulsion was cooled further to approximately 30° C., with stirring. Phase D and then phase Z were added during the cooling process at below 40° C.

### Example 7

### Anti-Dandruff Shampoo

| INCI name | % by weight |
|---|---|
| sodium laureth sulfate, 28% | 32.0% |
| fragrance | 0.3% |
| water | 57.5% |
| ether guanidine of example 1 | 0.2% |
| cocoamidopropylbetaine, 37.5% | 8.0% |
| PEG-18 glyceryl oleate/cocoate | 2.0% |
| NaCl | q.s. |
| preservative | q.s. |

Preparation: The constituents of anti-dandruff shampoo were mixed in succession with stirring, without heating.

### Example 8

### Conditioning Anti-Dandruff Shampoo

| INCI name | % by weight |
|---|---|
| phase A | |
| glycol distearate | 3.0% |
| sodium laureth sulfate, 28% | 40.0% |
| phase B | |
| fragrance | 0.3% |
| zinc pyrithione, 48% | 2.0% |
| quaternium-80 | 1.0% |
| phase C | |
| water | 36.6% |
| ether guanidine of example 1 | 0.3% |
| acrylates/C10-30 alkyl acrylates crosspolymer | 0.2% |
| polyquaternium-10 | 0.3% |
| NaOH, 10% | 0.3% |
| phase D | |
| undecyleneamidopropylbetaine | 12.5% |
| isostearamide MIPA | 3.5% |
| phase Z | |
| preservative | q.s. |

Preparation: Phase A was warmed to approximately 65° C. until the glycol distearate had melted, and the mixture was subsequently cooled to 45° C. Then, the constituents of phase B were added step by step to phase A in the order specified.

To prepare phase C, the carbomer was dispersed in water and stirred until swelling was complete. Thereafter, polyquaternium-10 was added, and the mixture was stirred until it, too, had swollen completely. Then, the mixture was neutralized using NaOH. Subsequently, phase C was added to the remainder of the formulation. Finally, the constituents of phase B and phase Z were added in succession.

### Example 9

### Clear Anti-Dandruff Shampoo

| INCI name | % by weight |
|---|---|
| sodium laureth sulfate, 28% | 32.0% |
| piroctone olamine | 0.5% |
| fragrance | 0.3% |
| dimethicone propyl pg-betaine | 0.5% |
| water | 56.3% |
| ether guanidine of example 1 | 0.2% |
| cocamidopropylbetaine 37.5% | 8.0% |
| cocamidopropylbetaine, glyceryl laurate | 2.2% |
| NaCl | q.s. |
| preservative | q.s. |

Preparation: The constituents of the formulation were mixed step by step in the order specified, without warming up.

### Example 10

### Conditioning Anti-Dandruff Shampoo for Highly Damaged Hair

| INCI name | % by weight |
|---|---|
| phase A | |
| sodium laureth sulfate, 28% | 32.0% |
| palmitamidopropyltrimonium chloride | 1.5% |
| PEG-100 hydrogenated glyceryl palmate; | 2.2% |
| PEG-7 glyceryl cocoate | |
| quaternium-80 | 2.0% |
| fragrance | 0.3% |
| phase B | |
| water | 54.9% |
| ether guanidine of example 1 | 0.3% |
| polyquaternium-10 | 0.3% |
| phase C | |
| cocamidopropylbetaine, 47% | 6.5% |
| phase Z | |
| NaCl | q.s. |
| preservative | q.s. |

Preparation: The constituents of phase A were mixed step by step. To prepare phase B, the ether guanidine was dissolved in water. Thereafter, the polyquaternium-10 was added, and the solution was stirred until swelling was complete. Thereafter, phase B was added to phase A. Finally, phase C and Z were added in succession.

### Example 11

### Clear Hair Tonic with Anti-Dandruff Activity

| INCI name | % by weight |
|---|---|
| PEG-40 hydrogenated castor oil | 1.0% |
| fragrance | 0.2% |
| quaternium-80 | 0.4% |
| dimethicone propyl PG-betaine | 0.6% |
| cetrimonium chloride | 0.3% |
| water | 82.25% |
| ether guanidine of example 1 | 0.05% |
| creatine | 0.2% |
| ethanol | 15.0% |
| preservative | q.s. |

Preparation: PEG-40 hydrogenated castor oil was warmed to 40° C. until it was clear. Then, the remaining constituents of the formulation were added step by step, without warming.

### Example 12

### Deodorant Spray with Propane/Butane

| INCI name | % by weight |
|---|---|
| cyclomethicone | 4.7% |
| PEG-8 | 4.7% |
| alcohol | 89.1% |
| polyglyceryl-3 caprylate | 1.3% |
| ether guanidine of example 1 | 0.2% |
| fragrance | q.s. |

aerosol filling:

30% solution

70% propellant

Preparation: The constituents of the formulation were mixed step by step in the stated order with stirring, without heating.

### Example 13

### Antiperspirant Spray

| INCI name | % by weight |
|---|---|
| phase A | |
| PEG-40 hydrogenated castor oil | 1.0% |
| fragrance | 0.3% |
| alcohol | 20.0% |
| phase B | |
| betaine | 2.0% |
| water | 56.3% |
| ether guanidine of example 1 | 0.3% |
| allantoin | 0.1% |
| aluminum zirconium tetrachlorohydrate, 35% | 20.0% |
| preservative | q.s. |

Preparation: Phase A was warmed to 50° C. Thereafter, phase B was added, without warming.

### Example 14

### Roll-On Deodorant

| INCI name | % by weight |
|---|---|
| phase A | |
| steareth-2 | 2.2% |
| stearath-20 | 1.0% |
| cetearyl ethylhexanoate | 2.0% |
| PPG-11 stearyl ether | 2.0% |
| dimethicone | 0.5% |
| zinc ricinoleate | 1.0% |
| phase B | |
| glycerol | 3.0% |
| water | 88.1% |
| ether guanidine of example 1 | 0.2% |
| phase Z | |
| preservative | q.s. |
| fragrance | q.s. |

Preparation: Phase A and B were warmed to 80° C., Then, phase B was added to A and the mixture was homogenized. Thereafter, the mixture was cooled to 30° C., with stirring, and phase Z was added.

### Example 15

### Clear Emollient Stick

| INCI name | % by weight |
|---|---|
| PPG-3 myristyl ether | 69.4% |
| propylene glycol | 13.5% |
| water | 4.0% |
| ether guanidine of example 1 | 0.1% |
| sodium stearate | 8.0% |
| cocamide DEA | 5.0% |
| preservative | q.s. |

Preparation: The constituents were mixed step by step in the stated order at 80° C. Then, the mixture was cooled to 75° C., with stirring, and the product was packaged into the stick sleeves.

### Example 16

### PEG-Free Antiperspirant Lotion

| INCI name | % by weight |
|---|---|
| phase A | |
| methyl glucose sesquistearate | 1.75% |
| polyglyceryl-4 laurate | 0.25% |
| diethylhexyl carbonate | 3.5% |
| ppg-14 butyl ether | 3.5% |
| polyglyceryl-3 caprylate | 0.5% |
| phase B | |
| water | 74.3% |
| ether guanidine of example 1 | 0.2% |
| hydroxyethylcellulose | 1.0% |
| phase C | |
| aluminum chlorohydrate | 15.0% |
| phase Z | |
| preservative | q.s. |
| fragrance | q.s. |

Preparation: Phase A and B were warmed to 70-75° C. Then, phase B was added to A and the mixture was homogenized. Thereafter, the mixture was cooled to 30° C., with stirring. Phase C was added during the cooling process at below 40° C.

### Example 17

### O/W Lotion for Wipes (Suitable for Acne Skin)

| INCI name | % by weight |
|---|---|
| phase A | |
| glyceryl stearate; ceteth-20 | 3.0% |
| stearyl alcohol | 1.0% |
| ethylhexyl stearate | 6.0% |
| C12-15 alkyl benzoate | 5.0% |
| phase B | |
| water | 81.7% |
| glycerol | 3.0% |
| phytosphingosin HCl | 0.2% |
| ether guanidine of example 1 | 0.1% |
| phase Z | |
| preservative | q.s. |
| fragrance | q.s. |

Preparation: Phase A and B were warmed to 80° C. Then, phase B was added to A and the mixture was homogenized. The emulsion was subsequently cooled to 30° C., with stirring.

### Example 18

### Impregnation Solution for Wipes

| INCI name | % by weight |
|---|---|
| polysorbate 80 | 3.0% |
| fragrance | 0.3% |
| bis-PEG/PPG-20/20 dimethicone | 0.5% |
| water | 91.4% |
| creatine | 0.5% |
| ether guanidine of example 1 | 0.2% |
| panthenol | 0.1% |
| propylene glycol | 4.0% |
| citric acid, 10% | q.s. |
| preservative | q.s. |

Preparation: The constituents of the formulation were mixed step by step in the stated order. Finally, the pH was brought to 6.5 with citric acid.

### Example 19

### Impregnation Solution for Face-Cleansing Wipes

| INCI name | % by weight |
|---|---|
| PEG-40 hydrogenated castor oil | 2.0% |
| fragrance | 0.2% |
| bis-PEG/PPG-20/20 dimethicone | 0.5% |
| disodium laureth sulfosuccinate | 2.5% |
| water | 93.4% |
| panthenol | 0.1% |
| sodium cocoamphoacetate | 1.0% |
| ether guanidine of example 1 | 0.3% |
| preservative | q.s. |

Preparation: PEG-40 hydrogenated castor oil was warmed to 40° C. until it was clear. Then, the remaining constituents of the formulation were added step by step in the stated order, with stirring.

1. A method of preventing and reducing the growth of undesired microorganisms comprising contacting a surface capable of exhibiting said microorganisms with at least one ether guanidine of general formula (I) 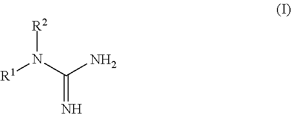
and/or of a salt or hydrate thereof, in which
R¹═—CH₂—CH₂—CH₂—O—R³, where R³ is a linear or branched hydrocarbon radical having 6 to 22 carbon atoms, and
R²═H or a hydrocarbon radical which has 1 to 22 C atoms,
to prevent or reduce the growth of microorganisms on said surface.

2. The method as claimed in claim 1, wherein R²═H.

3. The method as claimed in claim 1, wherein
R³ is a hydrocarbon radical having 12 to 15 carbon atoms.

4. The method as claimed in claim 1, wherein the at least one ether guanidine is used as the salt of at least one organic or inorganic acid selected from the group consisting of formic acid, acetic acid, propionic acid, butanoic acid, isobutanoic acid, hexanoic acid, heptanoic acid, octanoic acid, caprylic acid, nonanoic acid, capric acid, undecanoic acid, lauric acid, myristic acid, palmitic acid, stearic acid, arachic acid, behenic acid, cyclopentanecarboxylic acid, cyclohexanecarboxylic acid, acrylic acid, methacrylic acid, vinylacetic acid, isocrotonic acid, crotonic acid, 2-/3-/4-pentanoic acid, 2-/3-/4-/5-hexenoic acid, lauroleic acid, myristoleic acid, palmitoleic acid, oleic acid, gadoleic acid, sorbic acid, linoleic acid, linolenic acid, pivalic acid, ethoxyacetic acid, phenylacetic acid, glycolic acid, lactic acid, cinnamic acid, sorbic acid, nicotinic acid, urocanic acid, pyrrolidonecarboxylic acid, 2-ethylhexanoic acid, oxalic acid, glycolic acid, malic acid, malonic acid, succinic acid, tartaric acid, glutaric acid, citric acid, adipic acid, pimelic acid, suberic acid, azelaic acid, sebacic acid, maleic acid, fumaric acid, benzoic acid, o-/m-/p-toluic acid, o-/m-/p-hydroxybenzoic acid, salicylic acid, 3-/4-hydroxybenzoic acid, phthalic acid, terephthalic acid, hexahydro- or tetrahydrophthalic acid, glycine, alanine, beta-alanine, valine, leucine, phenylalanine, tyrosine, serine, threonine, methionine, cysteine, cystine, proline, hydroxyproline, pipecolic acid, tryptophan, aspartic acid, asparagine, glutamic acid, glutamine, lysine, histidine, ornithine, arginine, aminobenzoic acid, methanesulfonic acid, trifluoromethanesulfonic acid, benzenesulfonic acid, p-toluenesulfonic acid, carbonic acid, phosphoric acid, hydrochloric acid, hydrobromic acid, hydriodic acid, hydrofluoric acid, perchloric acid, nitric acid, sulfuric acid and mixtures thereof.

5. The method as claimed in claim 1, wherein a mixture of a plurality of ether guanidines of the general formula (I) are used.

6. The method as claimed in claim 1, wherein the microorganisms are Corynebacterium xerosis, Propionibacterium acnes, Staphylococcus epidermidis, Candida albicans, Streptococcus mutans, or Malassezia furfur.

7. A formulation comprising at least one ether guanidine of general formula (I) 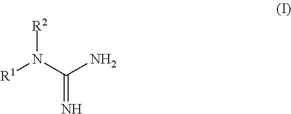
and/or of a salt or hydrate thereof, in which
R¹═—CH₂—CH₂—CH₂—O—R³, where R³ is a linear or branched hydrocarbon radical having 6 to 22 carbon atoms, and
R²═H or a hydrocarbon radical which has 1 to 22 C atoms.

8. The formulation as claimed in claim 7, wherein said at least one ether guanidine of general formula (I) is obtained by guanidylating an ether amine.

9. The formulation as claimed in claim 7, which comprises from 0.1 to 15% by weight of said at least one ether guanidine of general formula (I), based on the total formulation.

10. The formulation as claimed in claim 7, further comprising antitranspirant or deodorant agents.

11. (canceled)

12. A method for treating acne, comprising applying to skin at least one ether guandine of general formula (I) 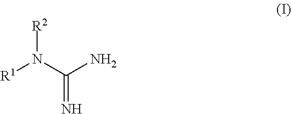
and/or of a salt or hydrate thereof, in which
R¹═—CH₂—CH₂—CH₂—O—R³, where R³ is a linear or branched hydrocarbon radical having 6 to 22 carbon atoms, and
R²═H or an optionally branched a hydrocarbon radical which has 1 to 22 C atoms and which optionally comprises double bonds,
to treat said acne.

13. The method as claimed in claim 12, wherein R²═H.

14. The method as claimed in claim 12, wherein R³ is a hydrocarbon radical having 12 to 15 carbon atoms.

15. The method as claimed in claim 12, wherein wherein the at least one ether guanidine is used as the salt of at least one organic or inorganic acid selected from the group consisting of formic acid, acetic acid, propionic acid, butanoic acid, isobutanoic acid, hexanoic acid, heptanoic acid, octanoic acid, caprylic acid, nonanoic acid, capric acid, undecanoic acid, lauric acid, myristic acid, palmitic acid, stearic acid, arachic acid, behenic acid, cyclopentanecarboxylic acid, cyclohexanecarboxylic acid, acrylic acid, methacrylic acid, vinylacetic acid, isocrotonic acid, crotonic acid, 2-/3-/4-pentanoic acid, 2-/3-/4-/5-hexenoic acid, lauroleic acid, myristoleic acid, palmitoleic acid, oleic acid, gadoleic acid, sorbic acid, linoleic acid, linolenic acid, pivalic acid, ethoxyacetic acid, phenylacetic acid, glycolic acid, lactic acid, cinnamic acid, sorbic acid, nicotinic acid, urocanic acid, pyrrolidonecarboxylic acid, 2-ethylhexanoic acid, oxalic acid, glycolic acid, malic acid, malonic acid, succinic acid, tartaric acid, glutaric acid, citric acid, adipic acid, pimelic acid, suberic acid, azelaic acid, sebacic acid, maleic acid, fumaric acid, benzoic acid, o-/m-/p-toluic acid, o-/m-/p-hydroxybenzoic acid, salicylic acid, 3-/4-hydroxybenzoic acid, phthalic acid, terephthalic acid, hexahydro- or tetrahydrophthalic acid, glycine, alanine, beta-alanine, valine, leucine, phenylalanine, tyrosine, serine, threonine, methionine, cysteine, cystine, proline, hydroxyproline, pipecolic acid, tryptophan, aspartic acid, asparagine, glutamic acid, glutamine, lysine, histidine, ornithine, arginine, aminobenzoic acid, methanesulfonic acid, trifluoromethanesulfonic acid, benzenesulfonic acid, p-toluenesulfonic acid, carbonic acid, phosphoric acid, hydrochloric acid, hydrobromic acid, hydriodic acid, hydrofluoric acid, perchloric acid, nitric acid, sulfuric acid and mixtures thereof of these.

16. The formulation of claim 7 comprising a pharmaceutical or cosmetic composition.

17. The method of claim 1 wherein said surface is human skin.
